(19) United States
(12) Patent Application Publication
(10) Pub. No.: US 2007/0161030 A1
(43) Pub. Date: Jul. 12, 2007

(54) Micelle-and microemulsion-assisted planar separations platform for proteomics

(75) Inventor: Wayne F. Patton, Newton, MA (US)

Correspondence Address: WILMER CUTLER PICKERING HALE AND DORR LLP, 60 STATE STREET, BOSTON, MA, 02109, US

(73) Assignee: PerkinElmer LAS, Inc.

(21) Appl. No.: 11/636,327

(22) Filed: Dec. 8, 2006

Related U.S. Application Data
(60) Provisional application No. 60/748,472, filed on Dec. 8, 2005.

(51) Int. Cl. C12Q 1/68; G01N 33/53

(52) U.S. Cl. 435/6; 435/7.1; 204/450

(57) ABSTRACT

A method of separating biomolecules includes providing a sample comprising one or more biomolecules, at least one of which is hydrophilic; wetting an amphiphilic planar stationary phase with a microemulsion or micelluar mobile phase; and creating an electrical field between first and second electrodes in electrical contact with opposing edges of the amphiphilic stationary phase, wherein the mobile phase advances across the length of the separation medium and one or more biomolecules are separated.

### CROSS-REFERENCE TO RELATED APPLICATIONS

The application claims benefit of U.S. provisional patent application No. 60/748,472, filed Dec. 8, 2005, the contents of which are incorporated by reference.

### BACKGROUND

The human proteome is known to contain approximately 30,000 different genes, but due to post-translational modifications and differential mRNA splicing, the total number of distinct proteins is most likely to be close to one million. The level of complexity, coupled with the relative abundances of different proteins, presents unique challenges in terms of separations technologies. One particularly significant challenge is that the human proteome contains both hydrophilic and hydrophobic proteins. The hydrophobic proteins are often more difficult to characterize by currently implemented analytical approaches. As a case in point, integral membrane proteins typically contain one or more hydrophobic, transmembrane domains that intermingle with the hydrophobic portion of lipid bilayer membranes. Given the prominent role that many integral membrane proteins play in signal transduction, they are considered important drug targets for the pharmaceutical industry.

Proteomic profiles generated from two-dimensional gel electrophoresis (2DGE) are known to lack highly hydrophobic proteins, particularly integral membrane proteins containing more than one alpha-helical transmembrane domain. This is thought to be due to the poor resolution of this class of proteins in the isoelectric focusing component of the procedure, arising from poor solubilization by nonionic detergents, even in the presence of high concentrations of urea. Should solubilization be achieved initially, the proteins then tend to subsequently precipitate near their isoelectric point. Because the technique relies upon aqueous buffers and hydrophilic polymers, 2DGE is unsuitable for fractionating this class of proteins.

Conventional sodium dodecyl sulfate (SDS)-polyacrylamide gel electrophoresis is in many ways more suitable for fractionating integral membrane proteins than 2DGE, due to the favorable solubilization properties of the anionic detergent. Should mobile phases rich in hydrophobic organic solvents be employed in protein separations, then the solubility of the hydrophilic components of the proteome are compromised.

Micellar electrokinetic capillary chromatography (MEEK) and microemulsion electrokinetic capillary chromatography (MEEKC) are electrodriven separation techniques, which can separate both charged and neutral species. The techniques use micelles or microemulsion buffers to separate solutes based on both their hydrophobicities and electrophoretic mobilities.

### SUMMARY

One aspect of the invention provides a method for simultaneously separating and analyzing a range of proteins from complex biological samples. The technology facilitates separation of the analytes by providing an appropriate microenvironment for solubilization of hydrophobic proteins, while still allowing the hydrophilic proteins to be maintained in a substantially aqueous-based environment.

In one aspect, a method of separating biomolecules is provided, including the steps of (a) providing a sample comprising one or more biomolecules, at least one of which is hydrophilic; (b) wetting an amphiphilic planar stationary phase with a microemulsion or micelluar mobile phase; and (c) creating an electrical field between first and second electrodes in electrical contact with opposing edges of the amphiphilic stationary phase, wherein the mobile phase advances across the length of the separation medium and one or more biomolecules are separated.

In one or more embodiments, the sample includes hydrophobic and hydrophilic proteins.

In one or more embodiments, the mobile phase includes micelles or a microemulsion, such as for example, an oil-in-water emulsion. In one or more embodiments, the microemulsion includes a surfactant-coated nanodroplet of a liquid selected from a hydrophobic liquid and a lipophilic liquid, and may further a co-surfactant or a hydrophobic tracker dye.

In one or more embodiments, the planar stationary phase comprises pores of about 0.1 microns to about 100 microns in diameter, the planar stationary phase comprises particles having a diameter of about 1 microns to about 10 microns.

In one or more embodiments, the pH, ionic strength, and water/organic content of the microemulsion or micellar mobile phase are selected to promote electroosmosis-driven separation.

In one or more embodiments, the method further includes generating a second electrical potential between the first electrode and the second electrode so as to cause a second liquid mobile phase to be advanced across the length of the stationary phase in a second direction, whereby one or more biomolecules are separated.

In one or more embodiments, the separation by advancing microemulsion or micelluar mobile phase across the length of the stationary phase occurs electrokinetically, and the separation by advancing second liquid mobile phase across the length of the stationary phase occurs chromatographically. The second liquid mobile phase can include a microemulsion or micelluar mobile phase. The second separation can be microemulsion thin layer chromatography.

In one or more embodiments, the method further includes detecting the separated biomolecules.

The detection is selected from the group consisting of fluorescence, mass spectrometry, chemiluminescence, radioactivity, evanescent wave, label-free mass detection, optical absorption and reflection, and for example, detection is selected from a group consisting of MALDI-TOF mass spectrometry, and inductively-coupled plasma mass spectrometry

In one or more embodiments, the biomolecules are labeled with a detection agent prior to separation, or the biomolecules are labeled with a detection agent after separation, such as colored dyes, fluorescent dyes, chemiluminescent dyes, biotinylated labels, radioactive labels, affinity labels, mass tags, and enzymes.

In one or more embodiments, the method further includes mass tagging the biomolecules for differential analysis of protein, or multiplexing for parallel determination of protein characteristics.

In another aspect, a kit for conducting micelle- or microemulsion-assisted planar electrochromatography is provided. The kit includes a separations plate comprising a planar amphiphilic separation medium having pores or connected pathways of a dimension that permits migration of proteins, and at least one electrode buffer solution suitable for use in planar electrochromatographic separations.

In one or more embodiments, the kit further includes an impermeable barrier to cover the separation medium, such as glass plate or silicone oil.

In one or more embodiments, the planar amphiphilic separation medium has a pore size in the range of 0.1 μm to 100 μm, or the planar amphiphilic separation medium has a pore size in the range of 1 μm to 10 μm.

### BRIEF DESCRIPTION OF DRAWINGS

The invention is described with reference to the following drawings, which are presented for the purpose of illustration only and are not intended to be limiting of the invention.

FIG. 1 is a schematic illustration of an exemplary microemulsion-encapsulated protein, consisting of a protein, an oil, an alcohol and a surfactant.

FIG. 2 is a schematic illustration of a planar electrochromatography apparatus that may be used in one or more embodiments of the invention.

### DETAILED DESCRIPTION

The technology described herein relates to planar separations methods using micellar and microemulsion materials, and related compositions and commercial packages. System and methods for separation of analytes, using micelle- or microemulsion-assisted electroosmosis-driven planar chromatography (also referred to as “micelle- or microemulsion-assisted PEC”) are described. The systems and methods described herein are useful in the separation of mixtures containing both ionic and neutral species or mixtures of hydrophilic and hydrophobic molecules. “Hydrophobic” molecules that are suitable for separation according to one or more embodiments of the invention include molecules such as proteins that contain at least a portion of the molecule that is hydrophobic or at least a protein of the molecule that is not readily soluble or does not significantly interact with water. Reference herein to micelle-assisted PEC or microemulsion-assisted PEC is considered to apply to both techniques unless other noted.

In one aspect, microemulsion-assisted electroosmosis-driven planar chromatography is used for the separation and detection of a biomolecule. An amphiphilic polymeric membrane, amphiphilic thin-layer chromatography plate or similar planar substrate provides the stationary phase for the separation platform. The planar substrate surface is characterized by a combination of charge carrying groups (ion exchangers), non-covalent charged groups (counterions), and nonionic groups that facilitate chemical interactions with the analyte, e.g., proteins or peptides. Electroosmotic flow is generated by application of a voltage across the planar support in the presence of a mobile phase. The mobile phase includes micelles or a microemulsion. Charged ions accumulate at the electrical double layer of the solid-phase support and move towards the electrode of opposite charge, dragging the liquid mobile phase along with them. Water-insoluble compounds will favor inclusion into the oil droplet (or interact with the micelles) rather than into the buffer phase. Separation of different analytes is the result of their differential association with the micelles (or microemulsion), the liquid mobile phase and the solid support.

The “amphiphilic stationary phase” refers to a solid-support stationary phase exhibiting both non-polar and polar interactions with the analyte, e.g., proteins. An amphiphilic stationary phase includes regions, phases or domains that are nonionic and/or hydrophobic in nature as well as regions, phases or domains that are highly polar and preferably ionic. The ionic regions can be positively or negatively charged. Hydrophobic groups favor the interaction and retention of the protein during separation, while the ionic groups promote the formation of the charged double layer used in electrokinetic separation. In one embodiment, the amphiphilic stationary phase for protein fractionation has a combination of charge carrying groups (ion exchangers), non-covalent groups, and nonionic groups that facilitate chemical interactions with the analytes. In another embodiment, the amphiphilic stationary phase is predominantly hydrophobic, but partially ionic in character.

In one aspect, micelles or microemulsions provide an appropriate microenvironment for solubilization of hydrophobic proteins, while still allowing the hydrophilic proteins to be maintained in a substantially aqueous-based environment. Hydrophobic proteins can be encapsulated in microemulsions having surfactant-stabilized nanometer-sized droplets that are suspended in aqueous-based buffer. A co-surfactant, such as a short-chain alcohol, can also be employed in order to stabilize the microemulsion. The co-surfactant typically has intermediate hydrophobicity relative to the oil and water phase and is thought to bridge the oil and water interface, thus reducing the surface tension of the system. With respect to the technology, both oil-in-water microemulsions and water-in-oil microemulsions can have application, based upon whether separations are performed in reverse-phase or normal-phase mode.

Surfactant systems are known to self-assemble into a variety of structures, ranging from the simplest structure, referred to as a micelle, to higher order structures such as microemulsions and liquid crystal phases. In aqueous solution, surfactants aggregate into structures called micelles. Micelles are closed shape structures in which the hydrophilic portions of the molecule are exposed to the surrounding water while the hydrophobic portions are protected from contact with water. When dissolved in oils, surfactants form reversed micelles in which the hydrophilic portions are shielded from contact with the surrounding solvent in the interior of the micelles. Depending on the particular molecular architecture of the surfactant molecule, a variety of microstructures can be formed, such as spherical, cylindrical and worm-like micelles.

Microemulsions are clear, thermodynamically stable, isotropic mixtures of oil, water and surfactant (e.g., a hydrophobic of lipophilic phase), frequently in combination with a co-surfactant. Typically, they are solutions containing dispersed droplets of a lipophilic organic solvent with diameters of 10 nm or less, though water-in-oil and bicontinuous microemulsions can also be created. Specific combinations of surfactant, oil and water are required to form microemulsions and this is commonly illustrated in the form of a ternary phase diagram of the multicomponent system, in which each corner of the diagram represents 100% of a particular component. Pseudo-ternary phase diagrams can also be constructed where a corner will typically represent a binary mixture of two components, such as oil/water or surfactant/co-surfactant. Contrary to conventional emulsions, microemulsions form spontaneously upon addition of appropriate amounts of a suitable surfactant or surfactant/co-surfactant combination to a mixture of oil and water. For oil-surfactant-water systems, two types of microemulsions predominate, oil-in-water and water-in-oil forms.

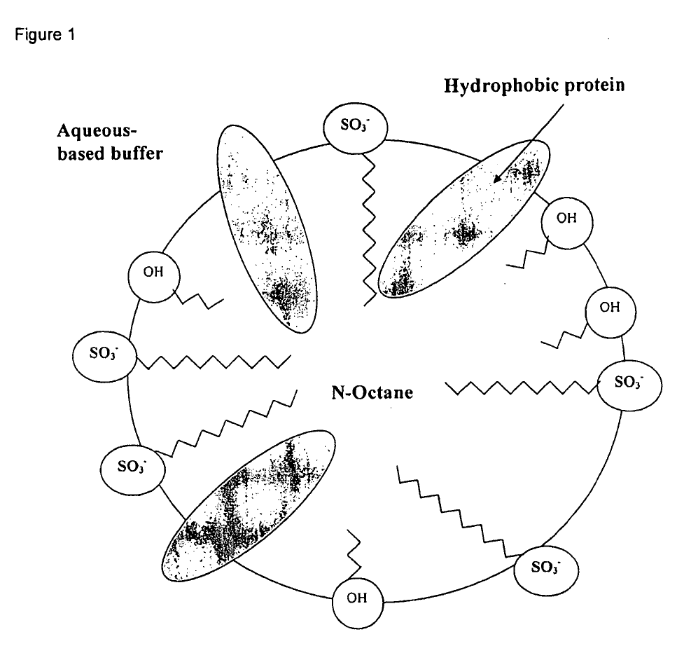

FIG. 1 is a schematic diagram illustrating the general principle of encapsulating hydrophobic proteins in an oil-in-water microemulsion for enhanced solubility prior to chromatographic or electrically-driven separation. A generic hydrophobic protein (grey) is shown in the specific example, however, the same technique applies equally to other hydrophobic macromolecules, such as lipoproteins, glycolipoproteins, lipopolysaccharides and various small molecules including lipids, glycolipids, phospholipids, steroids, saponins, glycosylphosphatidylinositols, gangliosides, isoprenoids, as well as various benzimidazole, benztriazole, phenol, and aniline derivatives. In the illustrated example, the microemulsion is formed from droplets of (A) n-octane or other lipophilic organic solvent (such as long chain alkane or alcohol), (B) a co-surfactant consisting of an alcohol of lower lipophilicity (such as butan-1-ol), (C) a charged surfactant (such as sodium dodecyl sulfate or cetylmethylammonium bromide), and (D) a hydrophobic protein. The colloidal droplets are suspended in an aqueous medium that can optionally contain a water-miscible more polar solvent, such as propan-2-ol. Hydrophilic proteins partition in the aqueous phase. The colloidal droplet serves two important functions. First, it provides an appropriate microenvironment for solubilization of the hydrophobic protein. Second, the charged groups on the surface of the droplet promote migration of the droplet during electrically-driven separation. The exact orientation and penetration of the protein in the microemulsion will vary on a case-by case basis, depending upon the primary, secondary and tertiary structure of the protein and is simply depicted in the diagram as a modest penetration at the oil-water interface. The components of the microemulsion are not drawn to scale in this illustration.

A large number of physiochemical parameters can be manipulated when developing protein-embedded micelles and microemulsions for use in chromatographic and electrically-driven planar separations. The type and concentration of the oil, surfactant, co-surfactant, buffer, organic solvent, counter-ion and pH will ultimately determine the nature of the micelle or microemulsion formed and its interaction with target molecules, such as proteins and peptides. Guidance for the creation of appropriate microemulsion systems for planar separations of proteins can be obtained from the drug delivery and capillary microemulsion electrokinetic chromatography (MEEKC) literature.

Microemulsions used in one or more embodiments of the invention may be optimized for high mobility upon application of an external electric field. Typically, pH 7-9 buffers are used with this electrophoretic technique in order to generate relatively high electroosmotic flow velocities. Also, typically very low ionic strength buffers are employed in order to minimize Joule heating during electrophoresis. Higher buffer concentrations would suppress endoosmotic flow and generate high currents which would limit the voltage that could be applied in the electrophoretic separation.

There is a wide range of choices for the mobile phase. With respect to using mobile phases for microemulsion-assisted separations based upon microemulsion drug delivery formulations, the intended application for protein separation alleviates many of the biomedical safety constraints that would otherwise be imposed, such as devising biocompatible or food-grade emulsions, and consequently allows a greater selection of raw materials that are generally less costly than those commonly used in the pharmaceutical industry, such as oleic acid or isopropyl palmitate (oils), PEG-40 hydrogenated castor oil or glyceryl oleate/polyoxyl 40 fatty acid derivatives (surfactants) and diethylene glycol monoethyl ether or tetraglycol (cosurfactants). Medium chain length alcohols are not generally used as co-surfactants in pharmaceutical applications, due to significant toxicity and irritancy issues, but this is generally not of concern for many applications of the technology described herein. In certain instances, however where a protein-based therapeutic or pharmaceutical is to be isolated and subsequently employed clinically, then biocompatible separation systems can be used.

Examples of lipophilic organic solvents and oils appropriate for application in microemulsion-assisted separations include n-octane, n-heptane, 1-chloropentane, 5-chloropentane, n-hexanol, diethyl ether, dibutyl ether, ethylene glycol diethyl ether, ethylene glycol dibutyl ether, cyclohexane, chloroform, methylene chloride, amyl alcohol, butyl chloride, ethyl acetate, octan-1-ol, octan-2-ol, hexan-1-ol, n-decane, n-decanol, soybean oil, fish oil, medium chain triglycerides (Miglycol 812), tributyrin, isopropyl myristate, isopropyl palmitate, cetyl 2-(hexylethyl)-2-hexanoate, ethyl 2-(hexylethyl)-2-hexanoate or ethyl oleate. Perfluorinated oils can also be used, but also require use of a fluorinated surfactant as otherwise segregation between the fluorinated and hydrogenated chains would occur, leading to heterogeneities or even gelation. Semi-fluorinated oils can effectively be combined with conventional hydrocarbon-based surfactants, however. Since the exact nature of the oil phase plays a minor role in the sequestering of the hydrophobic protein, a great degree of latitude in selecting the water-immiscible solvent is anticipated and oils containing 5-16 carbon atoms are suitable for the applications described herein.

Surfactant classes appropriate for micelle- and microemulsion-assisted separations include nonionic, cationic, anionic, or zwitterionic surfactants, as well as combinations of two or more of these classes. Fluorinated surfactants have some potentially interesting characteristics, such as critical micelle concentrations that are usually about two-orders of magnitude lower than their more commonly employed hydrocarbon counterparts. Examples of appropriate surfactants for the separations approach include sodium dodecyl sulfate (SDS), lithium dodecyl sulfate (LDS), sodium bis-2-ethylhexylsulphosuccinate, sodium cholate, perfluordecyl bromide, cetyltrimethylammonium bromide (CTAB), didodecylamonium bromide, Triton X-100, polyoxyethylene 10-oleyl ether, polyoxyethylene-10-dodecyl ether, N,N-dimethyldodecylamine-N-oxide, Brij 35, Tween-20, Tween-80, sorbitan monooleate, lecithin, diacylphosphatidylcholine, sucrose monolaurate and sucrose dilaurate. While a wide range of surfactants could be employed in microemulsion-assisted planar separations, SDS is a particularly good choice because oil droplets coated with this surfactant will confer a negative charge to the droplet, assisting in electrically-driven separations. SDS concentrations of 3 to 6.5% (w/w) are useful for the formation of stable microemulsions. Additionally, use of an acid labile mass spectrometry compatible surfactant is contemplated, particularly if the protein is to be subsequently analyzed by mass spectrometry. For example, a long-chain derivative of 1,3-dioxolane sodium propyloxy sulfate can be employed as a substitute for SDS. It should also be noted that hydrophilic and lipophilic linkers can be combined to form a self-assembled structure which has surfactant-like properties. For example sodium mono- and dimethyl naphthalene sulfonate can be employed as a hydrophilic linker while dodecanol can be used as the hydrophobic linker. The combination of these two molecules can largely replace the main surfactant while still retaining solubilization of the oil. Since the hydrophilic linker is adsorbed from the aqueous phase and the hydrophobic linker is localized in the oil phase, self-assembly occurs at the oil-water interface with this unusual class of surfactants.

Overall, the co-surfactant is characterized by a slightly lower hydrophobicity compared with the oil phase. A range of molecules can serve as co-surfactants, including nonionic detergents, alcohols, alkanoic acids, alkanediols and alkyl amines. Low molecular weight alcohols that are water-miscible, as well as alcohols and ethers with a higher degree of hydrophobicity and that are essentially insoluble in aqueous media can be employed to generate mobile phases for microemulsion-assisted planar separations of proteins. Branched alcohols, such as propan-2-ol and butan-2-ol are not well suited to serve as co-surfactants, while some exemplary co-surfactants include butan-1-ol and pentan-1-ol. However, certain branched alcohols, such as 2,4-dimethyl-3-pentanol and 3-ethyl-3-pentanol, can be successfully incorporated in such structures, if higher co-surfactant concentrations are employed. High concentrations of branched alcohols appear to be favorable due to their less efficient incorporation at the oil-water interface and the larger number of alcohol molecules per surfactant required to stabilize the microemulsions. The co-surfactant plays an important role in the formation and stability of the microemulsion, particularly when SDS is employed as the surfactant. Repulsive forces among the negatively charged sulfate head group on SDS molecules would otherwise prevent efficient packing and consequently formation of the emulsion.

Optionally, hydrophobic tracker dyes can be added to the micelles or microemulsions in order to monitor progress of planar separations. The dyes partition into of the hydrophobic layer of the micelles or the interior of microemulsions and simply serve as a colored or fluorescent marker during the planar separations. Alternatively, the dyes can be employed to permit visualization of the migrating proteins during the separation. Examples of lipophilic dyes suitable for the application include Nile Red dye, 1-anilinonaphthalene-8-sulfonic acid (ANS), 1,6-diphenyl-1,3,5-hexatriene (DPH), C11-BODIPY581/591, 10-N-nonyl acridine orange (NAO), 7-nitro-benz-2-oxa-1,3-diazole-(NBD)ceramide, 6-propionyl-2-[dimethyl-amino]naphthalene (PRODAN), pyrene, 6-palmitoyl-2-[[trimethylammoniumethyl]methylamino]naphthalene chloride (PATMAN), 2′-(N,N-dimethylamino)-6-naphthoyl-4-trans-cyclohexanioc acid (DANCA), 6-acetyl-2-(dimethylamino)naphthalene (Acdan), 6-lauroyl-2-(dimethylamino)naphthalene (Laurdan), Sudan Black B, Oil Red, Yellow OB, Orange OT, Butter Yellow and nigrosin. Some dye families especially relevant to the technology include carbocyanine dyes, styryl dyes, cyanine dyes, merocyanine dyes and oxonol dyes. The presence of microdomains that differ in polarity within the same single-phase solution permits both water-soluble and oil-soluble dyes or labels to be solubilized at the same time, if desired. Dyes or labels themselves can be amphiphilic and thus can influence the extent or even the existence of the microemulsion.

Examples of water-miscible organic additives appropriate for the application include acetonitrile, methanol, ethanol, isopropanol, propan-2-ol, glycerol, sorbitol and N-methyl-2-pyrrolidone. The latter family of additives serves as an organic modifier to impact distribution of the proteins by influencing their partitioning between the oil and water phase. This class of additive can be present in the aqueous phase in concentrations as high as 25% or more.

Micelles and microemulsions are relatively easy to prepare. The various materials are weighed out and mixed together, thus producing a cloudy suspension. Sonication of the suspension for approximately 30 minutes results in the suspension producing an optically clear solution. An alternate way of producing the suspension is to vortex mix the aqueous/surfactant solution with the oil and then add the co-surfactant drop wise until the optically clear solution is generated spontaneously. Another approach is to combine the surfactant, co-surfactant and oil together and then add the water-based buffer containing additional surfactant until an optically clear solution is generated. Typically, the protein is applied to the solid phase support, though it can be solubilized directly in the oil prior to creating the microemulsion prior to application of the sample to the solid-phase support. Some examples of microemulsion formulations that are suitable for fractionating proteins according to the technology include the following: (1.) 0.81% w/w n-octane, 3.31% w/w SDS, 6.61% w/w butan-1-ol, 89.27% 50 mM sodium acetate, pH 4.5. (2.) 0.8% w/w 1-chloropentane, 3.3% w/w LDS, 6.6% butan-1-ol, 89.3% of 5% v/v acetic acid. (3.) 0.8% w/w 5-chloropentane, 3.3% w/w SDS, 6.6% w/w butan-1-ol, 89.3% w/w sodium citrate, pH 4.0. (4.) 0.8% w/w n-octane, 6% w/w SDS, 6.6% w/w butan-1-ol, 20% w/w propan-3-ol, 66.6% w/w 50 mM phosphate buffer, pH 7.0.

In addition to the mobile phase, separation of proteins according to the technology requires an appropriately porous planar solid-phase substrate. A feature of the planar stationary phase includes pores or connected pathways of a dimension that permits unimpeded migration of the proteins. The stationary phase may possess a pore size and particle size suitable for separation of proteins. In one or more embodiments, pore size can range from about 0.1 μm to about 100 μm. In other embodiments, the pore size is about 1-10 μm in diameter. Particle size may be selected to achieve these pore sizes and typically range from 1-2 μm to 20 μm. Larger particles may be used if is results in pore sizes within the desired range. TLC plates formed from Kieselguhr are useful in this context. This naturally occurring base material consisting of amorphous silicic acid of fossil origin (also referred to as diatomaceous earth) is characterized by large pores of sub-micron to tens of micron diameter, e.g., about 0.1 μm to 100 μm. In contrast, pores in polyacrylamide gels have been estimated to range from 0.5-500 nm, depending upon matrix composition, while HPLC packing typically use 30 to 100 nm pore size materials for protein separations. Thus, the material is porous enough to act as a solid-phase for microemulsion-assisted planar separations of proteins. Additionally, the base material can readily be thin-film coated with, for example titania, in order to restrict the pore diameter. Since there are over 12,000 diatom structures known, porosities can also be varied based upon selecting the proper source of the material. Zeolitisation procedures provide a means of introducing ion-exchange functionalities to the Kieselguhr.

Various mesoporous and macroporous materials are also suitable solid-phase supports for micelle- and microemulsion-assisted planar separation of proteins, including glasses or silicon with pores in the 1 nm to tens of micron range. Mesoporous molecular sieves developed using liquid surfactant templates of varying chain length can be used in the application, such as, for example, Exxon Mobil Corporation's M41S family of mesoporous silica materials. This family includes MCM-41 with a hexagonal array of uni-directional pores, MCM-48 with a three-dimensional cubic pore structure and MCM-50 with unstable lamellar structure. Analogous supports can be fabricated from other inorganic materials, such as titanium dioxide, rhenium dioxide, zirconium oxide and alumino-phosphoro-vanadates.

Membranes, particulate thin-layer chromatography substrates, large pore mesoporous substrates, grafted gigaporous substrates, gel-filled gigaporous substrates, nonporous reversed phase packing material and polymeric monoliths are other solid-phase substrates that can be employed with microemulsion- and micelle-assisted protein separations. In one or more embodiments, a substantially non-porous stationary phase includes particles, e.g., silica particles, with appropriate binder. In such systems, interactions with binder at interstitial locations may provide chromatographic interactions.

Examples of amphiphilic stationary phase that can be used for protein separation includes hydrophobic planar support derivatized with sulfonic acid, sulfopropyl, carboxymethyl, phosphate, diethylaminoethyl, diethylmethylaminoethy, allylamine or quartenary ammonium residues or the like. Hydrophobic planar supports derivatized with sulfonic acid, sulfopropyl, carboxymethyl, or phosphate residues enable cathodic electroosmotic flow, while hydrophobic planar supports derivatized with diethylaminoethyl, diethylmethylaminoethy, allylamine or quartenary ammonium residues enable anodic electroosmotic flow.

Membranes include polymeric sheets, optionally derivatized to provide the amphiphilic character of the planar stationary phase. Exemplary hydrophobic membranes for membrane-based electrochromatography of proteins and peptides include Perfluorosulfonic Nafion® 117 membrane (Dupont Corporation), partially sulfonated PVDF membrane, sulfonated polytetrafluoroethylene grafted with polystyrene, polychlorotrifluoroethylene grafted with polystyrene, or the like. Sulfonation of polyvinylidene difluoride (PVDF) can be achieved by incubation with sulfuric acid at a moderately high temperature. The degree of sulfonation can be systematically varied, where ion exchange capability of 0.25 meq/g is considered as “moderate” sulfonation. In these membranes separation depends upon the electrostatic interaction of proteins with sulfonated residues in combination with hydrophobic interactions with aromatic residues in the substrate. At pH in the range from about pH 2.0 to about pH 11.0, the protonated primary amine groups on the proteins will interact with sulfonated residues on the membrane. This interaction is diminished at pH greater than about pH 11.0. Sulfonate residues will be protonated at a pH less than about pH 2.0 and will lead to a decline in the electroosmosis driving force of the separation.

In some embodiments, PVDF membranes, used for the isolation by electroblotting of proteins separated by gel electrophoresis, can be derivatized with cationic functional groups in order to generate an amphiphilic membrane (e.g., Immobilon-CD protein sequencing membrane (Millipore Corporation)). For example, PVDF membrane can be etched with 0.5 M alcoholic KOH and subsequently reacted with polyallylamine under alkaline conditions. As another example, PVDF membranes can be derivatized with diethylaminoethyl or quartenary ammonium residues.

In some embodiments, the membrane is unsupported. In other embodiments, the membrane is supported or semi-supported. For example, the membrane can be held between two rigid or semi-rigid holders in the form of frames with large openings in the center. The membrane may also be rigidly supported on a solid support, for example, a glass plate. Membranes may be substantially non-porous. In such instances, the mobile phase moves over the surface of the membrane. In other embodiments, the membrane may be porous, in which case the mobile phase moves through the pores and/or channels of the membrane. Separation occurs by preferential interactions of the proteins with the hydrophobic surfaces or the interstial surfaces of the membrane.

As another example, a planar stationary phase useful for separation of proteins include underivitized silica thin-layer chromatography plates or silica thin-layer chromatography plates derivatized with alkyl groups (e.g. C₃-C₁₈ surface chemistry), aromatic phenyl residues, cyanopropyl residues or the like. In these instances, the silanol groups (of silica) provide the ion exchange qualities of the amphiphilic support and can be deprotonated at a pH of 8, leading to electroosmosis and thereby providing the ion exchange qualities of the amphiphilic support. At pH below pH 3, there will be a reduction or elimination in electroosmosis. In some embodiments, both hydrophobic groups, e.g., alkyl, and charged groups, e.g., sulfonic acid, can be attached to the same silica particle. As a further example, a stationary phase support for the separation of peptides and proteins by planar electrochromatography includes a gamma-glycidoxypropyltrimethoxysilane sublayer attached to the silica support of a thin-layer chromatography plate. A sulfonated layer is then covalently affixed between the sublayer and an octadecyl top layer. For separation of proteins in the 10 and 100 kDa range using a silica-based stationary phase, it is expected that derivitization with C8 and C4 groups, respectively, may be used. Phenyl functionalities are slightly less hydrophobic than C4 functionalities and may be advantageous for the separation of certain polypeptides.

Micelle- or microemulsion-assisted PEC using the stationary phases, mobile phases and microemulsions and micellar systems as described herein can be used to separate mixtures containing a variety of compounds. In one aspect, separation of proteins on an amphiphilic stationary phase using microemulsion-assisted PEC is contemplated. Typically, protein samples are prepared by first dissolving the proteins in the mobile phase or a weaker solvent of lower ionic strength. The amphiphilic planar stationary phase is wetted by a mobile phase, for example, a microemulsion-based mobile phase. A small volume of a sample is dispensed or spotted for example, by hand, on top of the stationary phase, near the center of the stationary phase. Alliteratively, the protein can be spotted near an edge of the plate, especially when prior information regarding the migration direction of the proteins is known. In other embodiments, spotting is performed by dispensing the sample with a pipette, a piezo-electric dispensing tip, a solid or quill pin. Spotting may be located anywhere on the membrane and location may be determined, in part, by the anticipated direction and extent of electromigration of the species. In another embodiment, precise location in spotting can be achieved using a Multiprobe or Janus liquid handling robot (PerkinElmer) capable of automated spotting of single locations or array spotting. An electric field is applied across the stationary phase. The applied potential and the length across which the potential is applied characterize the magnitude of the electric field. Water-insoluble compounds will favor inclusion into the oil droplet (or interact with the micelles) rather than into the buffer phase. Separation of different analytes is the results of their differential association with the micelles (or microemulsion), the liquid mobile phase and the solid support.

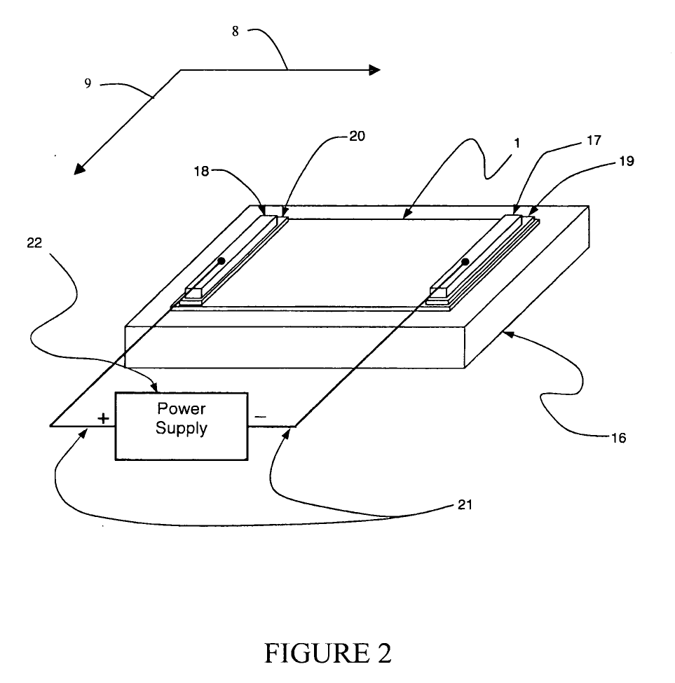

FIG. 2 is a schematic diagram of an apparatus for use in one or more embodiments of the invention. Referring to FIG. 2, planar stationary phase 1 is placed on a fixture or support 16 and a mobile phase (not shown) is applied to stationary phase 1. Support 16 may be solid, porous, or contain reservoirs or cavities to retain a supply of mobile phase to keep the stationary phase wet during separation. Exemplary support materials include PTFE (Teflon), Macor machineable ceramic, glass, or other compatible materials. Electrodes 17 and 18 are placed on top of stationary phase 1, with wire leads 21 connecting the electrodes to a power supply 22. In one embodiment, the electrodes are made of non-reactive metals. Exemplary non-reactive metals include platinum, palladium, or gold. The electrodes may be in the shape of rectangular bars, wires, rods, or any other shape with sufficient length to substantially span the width of the stationary phase. In one embodiment in accordance with the present invention, power supply 22 is a high-voltage DC supply. Power supply 22 may be controlled by a computer, a programmable controller, a microprocessor, a timer or the like in order to precisely control the separation conditions for more reproducible results. In some embodiments, connection pads 19 and 20 are placed between the electrodes and the stationary phase to ensure a continuous electrical connection along the entire lengths of electrodes 17 and 18. In one or more embodiments, planar stationary phase 1 can be rotated, e.g., by about 90 degrees, after a separation in first dimension 8 to facilitate another separation in second dimension 9. Further detail on the system and operation of a planar electrochromatography system is found in Published International PCT Appln. No. US05/009210, which is hereby incorporated in its entirety by reference.

In some embodiments, separation is conducted at elevated pressures, e.g., at pressures of greater than one atmosphere. Apparatus capable of planar electrochromatography at elevated pressures are described in U.S. Pat. Nos. 6,303,029 and 6,610,202 and United States Published Application Nos. 2004/0050763 and 2006/0175259, which are hereby incorporated in their entirety by reference.

Microemulsion-assisted thin-layer liquid chromatography (METLC) is a related technique in which a water-in-oil microemulsion is used as the mobile phase. As the mobile phase is not an electrically-driven separation, there is substantial opportunity to apply solvent conditions well outside of the realm of conventional MEEKC. Specifically, acidic pH and high ionic strength mobile phases can be applied in these separations. Increasing ionic strength will result in a decrease in the effective head group area of ionic surfactants, thus impacting microemulsion formation. This latitude in the chromatographic separation can be useful when devising orthogonal two-dimensional separations of proteins.

In two-dimensional separation of proteins on an amphiphilic stationary phase using 2D micelle- or microemulsion-assisted PEC/METLC, protein sample can be applied at the center of the solid phase support (dry or pre-wetted with mobile phase) and the planar stationary phase is then incubated in a microemulsion-based mobile phase. Alternatively, the protein can be spotted near an edge of the plate, especially when prior information regarding the migration direction of the proteins is known. Typically, protein samples are prepared by first dissolving the proteins in the mobile phase or a weaker solvent of lower ionic strength. The proteins are electrophoretically separated in one direction, as described above. Then, the planar stationary phase is evaporated or washed away, and the planar stationary phase is incubated in a second mobile phase. The protein sample then is chromatographically separated in a direction perpendicular to the first direction. Alternatively, the chromatographic separation is performed prior to the electrically-driven separation. Use of volatile hydrocarbons, fatty acids or oils in the microemulsion is desired, if the solvent is to be evaporated away prior to beginning the second dimension separation. Liquid mobile phases can be adjusted to different pH values, concentrations of organic solvent, and/or ionic strengths to facilitate 2D separations of proteins on the amphiphilic substrate. For example, one mobile phase will have acidic pH (ca. pH 4.5) and the other basic pH (ca pH 8.5). The pH of the buffers will affect the total charge of the individual protein species and thus influence their electrokinetic migration. Changes to the concentration of organic solvent in liquid mobile phase will impact the strength of interaction of the proteins with the hydrophobic component of the stationary phase. Finally, the ionic strength of the buffer will change the separation properties of the proteins in the two dimensions. By manipulating pH, ionic strength and organic solvent concentration, separation in one dimension will occur electrophoretically and separation in the other dimension will occur chromatographically. While both separations can be performed with microemulsion-based mobile phases, it is also possible to perform only one separation in a microemulsion-containing solvent and the other in a more conventional, liquid solvent. While 2D micelle- or microemulsion-assisted PEC/METLC is mentioned for illustrative purposes, planar micellar electrokinetic chromatography (MEKC) and planar micellar liquid chromatography (MLC) are alternative separation modalities that can be used together or in various combinations with either micelle- or microemulsion-assisted PEC or METLC.

Separations of protein, using the method in accordance with one or more embodiments of the present invention, can be achieved in a short duration. Proteins are spotted on a planar substrate, subjected to first dimension separation, rinsed and subjected to second dimension separation thereby providing access to the proteins and peptides on the surface of the stationary phase for detection. In one embodiment, fluorescamine is capable of detecting proteins on a surface within about 2 minutes. Additionally, the planar support itself serves as a mechanically strong support, allowing archiving of the separation profiles without the need for vacuum gel drying.

Proteins can be detected after microemulsion- or micelle-assisted PEC separation using a variety of detection modalities well known to those skilled in the art. Exemplary strategies employed for general protein detection include organic dye staining, silver staining, radio-labeling, fluorescent staining (pre-labeling, post-staining), chemiluminescent staining, mass spectrometry-based approaches, negative-staining approaches, contact detection methods, direct measurement of the inherent fluorescence of proteins, evanescent wave, label-free mass detection, optical absorption and reflection, and the like.

Protein samples that have undergone micelle- and microemulsion-assisted planar electrochromatography appear as discrete spots on the strip that are accessible to staining or immunolabeling as well as to analysis by various detection methods. Exemplary detection methods include mass spectrometry, Edman-based protein sequencing, or other micro-characterization techniques. In one embodiment, proteins bound to the surface of the membrane can be labeled by reagents, such as, antibodies, peptide antibody mimetics, oligonucleotide aptamers, quantum dots, Luminex beads or the like.

In some embodiments, chemiluminescence-based detection of proteins on planar surfaces can be used prior to or after fractionation by micelle- and microemulsion-assisted planar electrochromatography. In one embodiment, proteins can be biotinylated and then detected using horseradish peroxidase-conjugated streptavidin and the Western Lightning Chemiluminescence kit. In another embodiment, proteins may be fluorescently stained or labeled and the fluorescent dye subsequently chemically excited by nonenzymatic means, such as the bis(2,4,6-trichlorophenyl)oxalate (TCPO)—H₂O₂ reaction.

Micelle- and microemulsion-assisted protein separations can be probed by MALDI-TOF MS for direct analysis of proteins. For example, the microemulsion-assisted separation systems described herein can be used with an orthogonal MALDI-TOF mass spectrometer (e.g., PrOTOF 2000 PerkinElmer, Boston, Mass., USA/MDS Sciex, Concord, ON, Canada). The prOTOF 2000 MALDI O-TOF mass spectrometer is a MS MALDI with orthogonal time of flight technology. The prOTOF's novel design provides improved instrument stability, resolution, and mass accuracy across a wide mass range compared with conventional linear or axial-based systems. The more accurate and complete protein identification achieved with the prOTOF 2000 reduces the need for peptide sequencing using more complicated tandem mass spectrometry techniques such as Q-TOF and TOF-TOF. The instrument is particularly well suited for microemulsion-assisted protein separations because the MALDI source is decoupled from the TOF analyzer. As a result, any discrepancies arising from the solid phase surface topography or differential ionization of the sample from the surface are eliminated before the sample is actually delivered to the detector. The presentation of the proteins bound to a solid phase surface facilitates removal of contaminating buffer species and exposure to protein cleavage reagents (e.g., trypsin) prior to analysis by mass spectrometry.

In one application of the technology described herein, micelle- and microemulsion-assisted protein separations can be used with MALDI-TOF MS for direct analysis of proteins by providing proteins affixed to solid phase supports and thus suitably presented for direct probing by the MALDI-TOF laser. “Virtual” 3D profiles can be generated by 2D micelle- or microemulsion-assisted PEC/METLC separations, providing a charge- and hydrophobicity-based separation profile, followed by desorbing proteins directly from the planar substrate using MALDI-TOF mass spectrometry, providing a mass-based separation dimension. Analytical data obtained can be presented as a computer-generated image with 3D map appearance. In another aspect, 2D micelle- or microemulsion-assisted PEC/METLC can be used as a starting point for high throughput peptide mass fingerprinting and glycosylation analysis using chemical printing techniques such as piezoelectric pulsing where multiple chemical reactions are conducted on different regions of a spot by defined microdispensing of trypsin in-gel digestion procedures, and allowing peptide mass profiles and characterization of glycosylation, for example, to be achieved from the same spot. Defined microdispensing of trypsin and MALDI-TOF matrix solutions bypasses multiple liquid handling steps usually encountered with in-gel digestion procedures, and thus streamlines protein characterization methods.

In certain embodiments of the present invention, micelle- and microemulsion-assisted planar electrochromatography can be used to fractionate large proteins, small proteins, highly acidic proteins, highly basic proteins and hydrophobic proteins. In some embodiments, large multi-subunit complexes can be fractionated on the surface of a membrane. In one embodiment, mobile phases containing high concentrations of organic solvents are used to separate hydrophobic integral membrane proteins. In another embodiment, planar electrochromatography can be used to separate “electrophoretically silent” mutations, wherein proteins and peptides differ only by an uncharged amino acid residue. In a further embodiment, the planar electrochromatography system can be used to fractionate intact proteins. This is useful with respect to the analysis of protein isoforms arising from post-translational modification or differential splicing.

Proteomics studies are often based upon the comparison of different protein profiles. The central objective of differential display proteomics is to increase the information content of proteomics studies through multiplexed analysis. Currently, two principal gel-based approaches to differential display proteomics are being actively pursued, difference gel electrophoresis (DIGE) and Multiplexed Proteomics (MP). In one embodiment in accordance with the present invention, micelle- and microemulsion-assisted planar electrochromatography can be used with difference gel electrophoresis (DIGE) to increase the information content of proteomics studies through multiplexed analysis. Succinimidyl esters of the cyanine dyes (e.g., Cy2, Cy3 and Cy5) can be employed to fluorescently label as many as three different complex protein populations prior to mixing and running them simultaneously on the same 2D gel using DIGE. Images of the 2D gels are acquired using three different excitation/emission filter combinations, and the ratio of the differently colored fluorescent signals is used to find protein differences among the samples. DIGE allows two to three samples to be separated under identical electrophoretic conditions, simplifying the process of registering and matching the gel images. DIGE can be used to examine differences between two samples (e.g., drug-treated-vs-control cells or diseased-vs-healthy tissue). The principle benefit of the micelle- and microemulsion-assisted planar electrochromatography technology detailed in this disclosure with respect to DIGE is that protein separations can be achieved more quickly and samples are more readily evaluated by mass spectrometry after profile differences are determined. One requirement of DIGE is that from about 1% to about 2% of the lysine residues in the proteins be fluorescently modified, so that the solubility of the labeled proteins is maintained during electrophoresis. Very high degrees of labeling can be achieved when separations are performed by the micelle- and microemulsion-assisted planar electrochromatography technique, due to the fact that organic solvents are employed in the mobile phase and sample buffers. High degrees of labeling should in turn improve detection sensitivity using the DIGE technology.

In another embodiment, micelle- and microemulsion-assisted planar electrochromatography can be used with Multiplexed Proteomics to increase the information content of proteomics studies through multiplexed analysis. The Multiplexed Proteomics (MP) platform is designed to allow the parallel determination of protein expression levels as well as certain functional attributes of the proteins, such as levels of glycosylation, levels of phosphorylation, drug-binding capabilities or drug-metabolizing capabilities. The MP technology platform utilizes the same fluorophore to measure proteins across all gels in a 2DGE database, and employs additional fluorophores with different excitation and/or emission maxima to accentuate specific functional attributes of the separated species. With the MP platform, a set of 2D gels is fluorescently stained and imaged to reveal some functional attribute of the proteins, such as drug-binding capability, or a particular post-translational modification. Then, protein expression levels are revealed in the same gels using a fluorescent total protein stain. Differential display comparisons are made by computer, using image analysis software, such as Z3 program (Compugen, Tel Aviv, Israel). All gels are imaged using the same excitation/emission filter sets and resulting images are then automatically matched, with the option of adding some manual anchor points to facilitate the process. Any two images can then be re-displayed as a single pseudo-colored map. In addition, quantitative information can be obtained in tabular form, with differential expression data calculated. With a gel imaging platform similar profiles from different gels, such as total protein patterns, are matched by computer; while dissimilar ones from the same gel, such as total protein patterns and glycoprotein patterns, are superimposed and matched by computer. In MP the gels must be serially stained and imaged, as succeeding stains mask their predecessors in polyacrylamide gels. In one embodiment, micelle- and microemulsion-assisted planar electrochromatography can be used to assist MP in simultaneous imaging of multiple signals on profiles generated. Fluorescent dyes do not have the same strong tendency to mask one another on polymeric membranes.

In one or more embodiments, micelle- and microemulsion-assisted planar electrochromatography can be used with mass tagging techniques for differential display proteomics where relative abundances of different proteins in biological specimens are correlated with physiological changes. For example, Isotope-coded affinity tag (ICAT) peptide labeling is one such technique useful for distinguishing between two populations of proteins using isotope ratios. See, Gygi, S. P. et al. (1999) Quantitative analysis of complex protein mixtures using isotope-coded affinity tags. Nat Biotechnol 17 (10), 994-999. ICAT reagent employs a reactive functionality specific for the thiol group of cysteine residues in proteins and peptides. Two different isotope tags are generated by using linkers that contain either eight hydrogen atoms (d₀, light reagent) or eight deuterium atoms (d₈, heavy reagent). A reduced protein mixture from one protein specimen is derivatized with the isotopically light version of the ICAT reagent, while the other reduced protein specimen is derivatized with the isotopically heavy version of the ICAT reagent. Next, the two samples are combined, and proteolytically digested with trypsin or Lys-C to generate peptide fragments. The combined sample can be fractionated by micelle- and microemulsion-assisted planar electrochromatography. The ratio of the isotopic molecular weight peaks that differ by 8 daltons, as revealed by mass spectrometry, provides a measure of the relative amounts of each protein from the original samples. Other mass tagging approaches include growth of cells in either ¹⁴N— or ¹⁵N-enriched medium, use of regular water (H₂¹⁶O) and heavy water (H₂¹⁸O) as the solvent during Glu-C proteolysis of samples, use of acetate (d₀) and trideuteroacetate (d₃) to acetylate primary amino groups in peptides, methyl esterification of aspartate and glutamate residues using regular methanol (d₀) or trideuteromethanol (d₃), ¹²C and ¹³C labeled tri-alanine peptides iodoacetylated on their N-termini, and use of 1,2-ethanedithiol (d₀) and tetraalkyl deuterated 1,2-ethanedithiol (d₄) to measure differences between O-phosphorylation sites in samples using beta-elimination chemistry.

Recently, it has been demonstrated that the features of 2DGE and ICAT labeling technology can be combined into a single differential display platform. Proteins from two different samples are labeled with heavy and light. ICAT reagents, combined and then separated by 2DGE. The gel-separated proteins are detected with a sensitive protein stain, excised, treated with protease and identified by peptide mass profiling. Additionally, selected peptides can be evaluated further using collision-induced dissociation (CID) and sequence database searching. One important application of ICAT differential display in 2D gels is for the assessment of the relative abundances of protein isoforms that arise from post-translational modification. In one embodiment of the present invention, 2D planar electrochromatography can be combined with ICAT labeling into a single platform for differential display proteomics using the ICAT reagents. Separations are much faster and the proteins are more amenable to downstream mass spectrometry-based analysis.

Mass tagging approaches based upon the same basic principles as the ICAT strategy include growth of cells in either ¹⁴N- or ¹⁵N-enriched medium, and the use of regular water (H₂¹⁶O) and heavy water (H₂¹⁸O) as the solvent during Glu-C proteolysis of samples, leading to the incorporation of two 180 or two 160 atoms in the C-terminal moiety of each proteolytic fragment. This results in a 4 dalton difference in mass between paired peptides. Acetate (d₀) and trideuteroacetate (d₃) can be employed to acetylate primary amino groups in peptides. Similarly, methyl esterification of aspartate and glutamate residues using regular methanol (d₀) or trideuteromethanol (d₃) can be used as an isotope tagging strategy. ¹²C and ¹³C labeled tri-alanine peptides iodoacetylated on their N-termini for mass tagging experiments. Finally, 1,2-ethanedithiol (d₀) and tetraalkyl deuterated 1,2-ethanedithiol (d₄) can be employed to measure differences between O-phosphorylation sites in samples using beta-elimination chemistry. The pendant sulfhydryl group is then reacted with biotin iodoacetamidyl-3,6-dioxaoctanediamine. In one embodiment of the present invention, 2D planar electrochromatography can be used with mass tagging technologies as a separation platform for differential analysis of protein expression changes and post-translational modification changes.

In one or more embodiments, micelle- and microemulsion-assisted planar electrochromatography can be used with inductively-coupled plasma mass spectrometry (ICP-MS) for the trace elemental analysis of metalloproteins, such as selenoproteins, zinc metalloenzymes, cadmium-binding proteins, cisplatin-binding drug targets, and myoglobins subsequent to fractionation by planar electrochromatography. Laser ablation ICP-MS permits trace element analysis by combining the spatial resolution of an ultraviolet laser beam with the mass resolution and element sensitivity of a modern ICP-MS. UV laser light, produced at a wavelength of 193-266 nm is focused on a sample surface, causing sample ablation. Ablation craters of 15-20 microns are routinely produced by the instrumentation. No special sample preparation is required for the procedure. Ablated material is transported in an argon carrier gas directly to the high temperature inductively-coupled plasma and the resulting ions are then drawn into a mass spectrometer for detection and counting. A mass filter selects particles on the basis of their charge/mass ratio so that only specific isotopes are allowed through the filter and can enter the electron multiplier detector mounted at the end of the mass spectrometer (quadrupole, magnetic sector or time-of-flight instrumentation). Detected signals of individual isotopes can be converted to isotopic ratios or, when standards are measured along with the unknowns, to the actual element concentrations.

Laser ablation ICP-MS can be used for directly measuring phosphorous as m/z 31 signal liberated from phosphoproteins on electroblot membranes. Using Laser ablation ICP-MS, 16 pmole of the pentaphosphorylated beta-casein can be detected on polymeric membranes. In another embodiment, planar electrochromatography can be used as a platform for the direct analysis of protein phosphorylation, without the use of radiolabels or surrogate dyes, such as Pro-Q Diamond phosphoprotein stain (Molecular Probes).

The detection of low concentrations of phosphorous presents certain analytical challenges for ICP-MS due to its poor ionization in the argon ICP and the presence of interfering polyatomic species directly at mass 31 (¹⁵N¹⁶O and ¹⁴N¹⁶O¹H) and indirectly at mass 32 (¹⁶O₂ and ³²S). Phosphorous has a high first ionization potential of 10.487 electron volts (Wilbur and McCurdy, 2001). This translates to a poor conversion of phosphorous (P) atoms to P+ ions in the inductively coupled plasma. In a well-optimized ICP-MS, this translates to a 6% conversion of P atoms to P+ ions, a relatively low response factor for ICP-MS. It is known to one skilled in the art that phosphate groups in proteins and peptides readily bind certain trivalent metal ions, such as aluminum (III), gallium (III) and iron (III). Using ICP-MS, as little as 1 part per billion (ppb) of these metal ions can be detected. The ionized conversion of aluminum, which has a first ionization potential of 5.986 electron volts, is 99% under identical run conditions as described for phosphorous. Thus, detecting aluminum instead of phosphorous improves detection 16-fold. In addition, the specific detection of the trivalent metal ions shifts the detection window away from the cited biological background signal. The atomic masses of aluminum, gallium and iron are 26.98, 69.7 and 55.85, respectively. Among these three trivalent metal ions, the ferric ion poses problems due to polyatomic interferences arising from ArN, ArO and ArOH at the interface region of the ICP-MS. Gallium is probably the most suitable metal ion for the proposed application. Both ⁶⁹Ga, and ⁷¹Ga signal could be quantified by the method, minimizing the probability of overlapping signal from other molecular species.

The detection of proteins using ICP-MS-based detection procedure includes the following steps. First, proteins are separated by 2D micelle- and microemulsion-assisted planar electrochromatography as described in accordance with one embodiment of the present invention. The planar substrates are then incubated with 1 mM gallium chloride, 50 mM sodium acetate, pH 4.5, 50 mM magnesium chloride. Next, the planar substrates are washed repeatedly in 50 mM sodium acetate, pH 4.5, 50 mM magnesium chloride to remove excess metal ions. The individual spots on the planar surface are subjected to laser ablation ICP-MS methods where gallium signal is quantified rather than the phosphorous signal. Alternatively, the phosphorous signal can be read without incubating in the gallium solution. Sampling can be performed by single or multi-spot analysis, straight line scans or rastering. To aid in spot selection, the proteins on the planar substrate can be stained with a total protein stain, prior to the incubation with the gallium ions.

In one or more embodiments, micelle- and microemulsion-assisted planar electrochromatography can be used with protein microarrays for protein expression profiling and studying protein function. Micelle- and microemulsion-assisted planar electrochromatography can be used to provide a relatively simple method for generating protein microarrays. Small planar surfaces may be spotted with a defined mixture of proteins that are subsequently fractionated by 2D planar electrochromatography. Though the constituent proteins are not explicitly assigned a pre-determined coordinate in the resulting orthogonal matrix of spots thus generated, the identities of the spots can simply be determined by mass spectrometry, by immunodetection or by systematic omission of each protein from the mixture in subsequent separations. Once the location of each protein in the profile is known, the array may be used as conventional protein arrays, such as for profiling autoantibody responses in autoimmune disease and screening for other protein-protein, receptor-ligand, enzyme-substrate, enzyme-inhibitor or even protein-DNA interactions. In the arraying approach, a dedicated pin-based or piezoelectric spotting device is not required and the membrane arrays are amenable to filter-based protein microarray techniques as described recently. For example, a filtration approach that allows multi-stacking of protein chips can be used for simultaneously probing with a particular reagent.

In one or more embodiments, micelle- and microemulsion-assisted planar electrochromatography can be used for examination of biomarkers associated with specific proteins present in plasma, urine, lymph, sputum and other biological fluids. Serum albumin in particular is a high abundance blood protein with broad binding capability that serves as a depot and transport protein for numerous exogenous and endogenous circulating compounds. Once plasma is fractionated into its constituent serum protein components using methods described in this invention, peptides associated with discrete proteins, such as albumin, haptoglobin, α₂-macroglobulin or immunoglobulin, may be selectively eluted and identified by mass spectrometry. The peptides can be acid eluted with 0.2% trifluoroacetic acid and can subsequently be concentrated using reversed phase resin prior to analysis. Using this technique, noncovalently bound peptides can be isolated from a variety of proteins, such as hsp 70, hsp 90 and gp 96. In one aspect, the method obviates the need for separating the peptides from the protein by a molecular weight cut-off membrane. Instead, the target protein remains affixed to the electrochromatography substrate and the peptides are eluted away from it.

In one or more embodiments, micelle- and microemulsion-assisted planar electrochromatography can be used for the fractionation of complex oligosaccharides, glycoproteins, glycolipids, proteoglycans, and oligosaccharides pre-derivatized with fluorophores (such as 8-aminonaphthalene-1,3,6-trisulfonic acid (ANTS) and 2-aminoacridone (AMAC)). Protein glycosylation is used for biochemical alterations associated with malignant transformation and tumorogenesis. Glycosylation changes in human carcinomas contribute to the malignant phenotype observed downstream of certain oncogenic events. Technologies that permit the rapid profiling of glycoconjugate isoforms with respect to oligosaccharide branching, sialyation and sulfation are invaluable tools in assessing the malignant nature of clinical cancer specimens.

The following references are incorporated herein in their entirety: Altria K D. Background theory and applications of microemulsion electrokinetic chromatography. J Chromatogr A. 2000 Sep. 15;892(1-2):171-86; Altria K D, Mahuzier P E, Clark B J. Background and operating parameters in microemulsion electrokinetic chromatography. Electrophoresis. 2003 January;24(3):315-24; Bagwe R P, Kanicky J R, Palla B J, Patanjali P K, Shah D O. Improved drug delivery using microemulsions: rationale, recent progress, and new horizons. Crit Rev Ther Drug Carrier Syst. 2001; 18(1):77-140; Constantinides PP. Pharm Res. 1995 November; 12(11): 1561-72; Kreilgaard M. Influence of microemulsions on cutaneous drug delivery. Adv Drug Deliv Rev. 2002 Nov. 1;54 Suppl 1:S77-98; Lawrence M J. Surfactant systems: microemulsions and vesicles as vehicles for drug delivery. Eur J Drug Metab Pharmacokinet. 1994 July-September;19(3):257-69; Lawrence M J, Rees G D. Microemulsion-based media as novel drug delivery systems. Adv Drug Deliv Rev. 2000 Dec. 6;45(1):89-121; Tenjarla S. Microemulsions: an overview and pharmaceutical applications. Crit Rev Ther Drug Carrier Syst. 1999;16(5):461-521.

Although specific examples have been illustrated and described herein, it will be appreciated by those skilled in the art that any arrangement which is determined to achieve the same purpose can be substituted for the specific examples shown. This application is intended to cover any adaptations or variations of the technology described herein.

What is claimed is:

1. A method of separating biomolecules comprising the steps of:
providing a sample comprising one or more biomolecules, at least one of which is hydrophilic;
wetting an amphiphilic planar stationary phase with a microemulsion or micelluar mobile phase; and
creating an electrical field between first and second electrodes in electrical contact with opposing edges of the amphiphilic stationary phase, wherein the mobile phase advances across the length of the separation medium and one or more biomolecules are separated.

2. The method of claim 1, wherein the mobile phase comprises micelles.

3. The method of claim 2, wherein the mobile phase comprises a microemulsion.

4. The method of claim 3, wherein the microemulsion is an oil-in-water emulsion.

5. The method of claim 1, wherein the sample comprises hydrophobic and hydrophilic proteins.

6. The method of claim 1, wherein the microemulsion comprises a surfactant-coated nanodroplet of a liquid selected from a hydrophobic liquid and a lipophilic liquid.

7. The method of claim 6, wherein the liquid is selected from the group consisting n-octane, n-heptane, 1-chloropentane, 5-chloropentane, n-hexanol, diethyl ether, dibutyl ether, ethylene glycol diethyl ether, ethylene glycol dibutyl ether, cyclohexane, chloroform, methylene chloride, amyl alcohol, butyl chloride, ethyl acetate, octan-1-ol, octan-2-ol, hexan-1-ol, n-decane, n-decanol, oleic acid, soybean oil, fish oil, medium chain triglycerides (Miglycol 812), tributyrin, isopropyl myristate, isopropyl palmitate, cetyl 2-(hexylethyl)-2-hexanoate, perfluorinated oils, semi-fluorinated oils, ethyl 2-(hexylethyl)-2-hexanoate and ethyl oleate.

8. The method of claim 6, wherein the surfactant is selected from the group consisting of PEG-40 hydrogenated castor oil, glyceryl oleate/polyoxyl 40 fatty acid derivatives, sodium dodecyl sulfate (SDS), lithium dodecyl sulfate (LDS), sodium bis-2-ethylhexylsulphosuccinate, sodium cholate, perfluordecyl bromide, cetyltrimethylammonium bromide (CTAB), didodecylamonium bromide, Triton X-100, polyoxyethylene 10-oleyl ether, polyoxyethylene-10-dodecyl ether, N,N-dimethyldodecylamine-N-oxide, Brij 35, Tween-20, Tween-80, sorbitan monooleate, lecithin, diacylphosphatidylcholine, sucrose monolaurate, sucrose dilaurate, 1,3-dioxolane sodium propyloxy sulfate, and sodium mono- and dimethyl naphthalene sulfonate.

9. The method of claim 6, wherein the microemulsion further comprises a co-surfactant.

10. The method of claim 9, wherein the co-surfactant is selected from the group consisting of diethylene glycol monoethyl ether, tetraglycol, nonionic detergents, alcohols, alkanoic acids, alkanediols and alkyl amines.

11. The method of claim 6, wherein the microemulsion further comprises a hydrophobic tracker dye.

12. The method of claim 1, wherein the planar stationary phase comprises pores of about 0.1 microns to about 100 microns in diameter.

13. The method of claim 1, wherein the planar stationary phase comprises particles having a diameter of about 1 microns to about 10 microns.

14. The method of claim 1, wherein the pH, ionic strength, and water/organic content of said microemulsion or micellar mobile phase are selected to promote electroosmosis-driven separation.

15. The method of claim 1, further comprising the step of:
generating a second electrical potential between the first electrode and the second electrode so as to cause a second liquid mobile phase to be advanced across the length of the stationary phase in a second direction, whereby one or more biomolecules are separated.

16. The method of claim 15, wherein the separation by advancing microemulsion or micelluar mobile phase across the length of the stationary phase occurs electrokinetically, and wherein separation by advancing second liquid mobile phase across the length of the stationary phase occurs chromatographically.

17. The method of claim 16, wherein the second liquid mobile phase comprises a microemulsion or micelluar mobile phase.

18. The method of claim 16, wherein the second separation comprises microemulsion thin layer chromatography.

19. The method of claim 1, further comprising the step of detecting the separated biomolecules.

20. The method of claim 19, wherein detection is selected from the group consisting of fluorescence, mass spectrometry, chemiluminescence, radioactivity, evanescent wave, label-free mass detection, optical absorption and reflection.

21. The method of claim 19, wherein detection is selected from a group consisting of MALDI-TOF mass spectrometry, and inductively-coupled plasma mass spectrometry

22. The method of claim 1, wherein the biomolecules are labeled with a detection agent prior to separation.

23. The method of claim 1, wherein the biomolecules are labeled with a detection agent after separation.

24. The method of claim 23, wherein said detection agent is selected from the group consisting of colored dyes, fluorescent dyes, chemiluminescent dyes, biotinylated labels, radioactive labels, affinity labels, mass tags, and enzymes.

25. The method of claim 1, further comprising the step of mass tagging said biomolecules for differential analysis of protein.

26. The method of claim 1, further comprising the step of multiplexing for parallel determination of protein characteristics.

27. A kit for conducting micelle- or microemulsion-assisted planar electrochromatography, the kit comprising:
a separations plate comprising a planar amphiphilic separation medium having pores or connected pathways of a dimension that permits migration of proteins, and
at least one electrode buffer solution suitable for use in planar electrochromatographic separations.

28. The kit of claim 27, further comprising an impermeable barrier to cover said separation medium, wherein said impermeable barrier is glass plate or silicone oil.

29. The kit of claim 27, wherein the planar amphiphilic separation medium has a pore size in the range of 0.1 μm to 100 μm.

30. The kit of claim 27, wherein the planar amphiphilic separation medium has a pore size in the range of 1 μm to 10 μm.
